(19) United States
(12) Patent Application Publication
(10) Pub. No.: US 2006/0147904 A1
(43) Pub. Date: Jul. 6, 2006

(54) Use of resveratrol to regulate expression of apolipoprotein A1

(75) Inventor: Norman C.W. Wong, Calgary (CA)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Resverlogix, Inc.

(21) Appl. No.: 11/375,628

(22) Filed: Mar. 15, 2006

Related U.S. Application Data
(63) Continuation of application No. 10/222,013, filed on Aug. 15, 2002.

(51) Int. Cl. C12Q 1/00; C12Q 1/68; A61K 48/00

(52) U.S. Cl. 435/4; 435/6; 514/44

(57) ABSTRACT

Described are new methods for promoting the expression of apolipoprotein A1 (APO A1) for increasing levels of HDL, and assays for screening and identifying compounds for regulating expression of the APO A1 protein.

### FIELD OF INVENTION

The present invention describes a method of promoting the expression of a serum protein called apolipoprotein A1 (APO A1) and for screening compounds for regulating expression of APO A1 protein.

### BACKGROUND OF INVENTION

Resveratrol (trans-3,5,4′-trihydroxystilbene) is a natural polyphenol found in certain plants and berries including red grapes, raspberries, mulberries, peanuts and some other plants. It has been suggested that resveratrol, its metabolites and related polyphenols present in red wine may underlie an epidemiologic observation termed the “French Paradox”. This paradox relates to the finding of a low incidence of cardiovascular disease (CVD) in the French population despite the consumption of a diet containing a high content of saturated fat comparable to that in the North American population. The content of saturated fat in the North American diet is a major contributor to the incidence of ischemic heart disease. In France, however, a comparable diet is associated with an incidence of ischemic heart disease equal to ⅓ of that in the North American population. It has been speculated that resveratrol may contribute to the paradox comes from its potential role as an antioxidant and additionally, as yet unknown mechanism(s) of action. Resveratrol and related compounds are found in abundance in nature and one of the best known sources are the skins of red grapes, which can contain 50-100 micrograms, μg per gram (Jang et al., 1997) of skin. Resveratrol is found in many red wines and may also be obtained in commercial preparations.

In part the actions of resveratrol may arise from its suspected antioxidant properties that inhibit lipid peroxidation of low-density lipoprotein (LDL) particles and thus prevent the cytotoxicity of oxidized LDL. Increased abundance of oxidized LDL is a risk factor for developing CVD (Frankel et al., 1993; Chanvitayapongs et al., 1997). Platelet aggregation in the pathogenesis of CVD occurs at early and late stages of the disease including the final insult of arterial thrombosis. This is usually the terminal event leading to ischemia or myocardial infarction. Thus the ability of resveratrol to inhibit this platelet activity is thought to possibly help in both prevention of atherosclerosis (Rotondo et al., 1998; Soleas et al., 1997) and the final insult. These effects of resveratrol may comprise in part the cardioprotective effects of moderate amounts of red wine consumption.

The World Health Organization (WHO) defines CVD as the term used for a group of disorders of the heart and blood vessels including hypertension, coronary heart disease, cerebrovascular disease (stroke), peripheral vascular disease, heart failure, rheumatic heart disease, congenital heart disease and cardiomyopathies. CVD is also the leading cause of death in the general population and especially in those with diabetes mellitus. The WHO estimates that roughly ⅓^{rd} of deaths worldwide are due to CVD and a comparable value at 37% in North America, a figure that exceeds deaths from cancer by more than 10%. One of the modifiable risk factors that give rise to CVD is an elevated level of cholesterol. Cholesterol in the body is synthesized de novo in all cells but it may also come from dietary intake. Abnormally high levels of cholesterol can be the reasoning behind the development of ischemic heart disease, cerebrovascular disease and other disease states under the grouping of CVD.

Cholesterol in the blood does not exist in a free form because it has poor solubility in aqueous solutions. In the blood, lipids such as cholesterol are carried on lipoprotein particles. These lipid carriers are comprised of both protein and lipids. This combination gives rise to particles and serves the purpose of overcoming the inherent insolubility of the lipids. These lipoprotein particles may be divided into “good cholesterol” (high-density lipoproteins; HDL) or “bad cholesterol” (LDL). Numerous epidemiologic studies have shown that decreased levels of HDL are associated with an increased risk of CVD and that elevated levels of HDL or APO A1 has the opposite effect and lead to cardioprotection. Transgenic animals that over express human APO A1 protein have decreased numbers of atherosclerotic lesions in the vessels. In contrast, increased levels of LDL, especially in the oxidized form, are associated with an increased risk of CVD. Why HDL has beneficial effect on CVD stems from its role in a normal physiologic process whereby excess cholesterol is “shuttled” from extra-hepatic tissues to the liver for further metabolism and eventual excretion as bile acids or free cholesterol (Miller et al., 1985; Franceschini et al., 1991). This process is called Reverse Cholesterol Transport (RCT) and enhanced actions of RCT will lower the total level of cholesterol in the body. The major component of HDL is a 28 kDa protein, APO A1. Roughly 70% of the total protein component of HDL is comprised of APO A1 and the abundance of this protein predicts the amount of HDL in the blood. APO A1 alone or as part of HDL has anti-atherogenic properties (Miller 1987; Barter & Rye, 1996; Lucoma 1997). This feature is likely responsible for the inverse correlation between levels of APO A1/HDL and the risk of CVD. Patients showing elevated levels of APO A1/HDL have a decreased risk of CVD regardless of the total cholesterol level.

Increased levels of APO A1 are found in pre-menopausal women, can be induced with regular exercise, and moderate consumption of alcohol, in particular red wine (reviewed in Hargrove et al., 1999). Beyond these known factors, there is little in terms of pharmacologic agents that specifically raise the levels of the protein or HDL.

It is one aspect of the present invention to provide novel tools, reagents, methods and compounds to raise these levels of APO A1 and thus ADL.

Previous studies by the inventor have shown that thyroid hormone increases the level of APO A1 gene transcription. In addition, a thyromimetic, CGS23425 (Novartis), has a similar effect. Our results showed that both APO A1 protein levels and gene activity increase because of a direct effect induced by the binding of these ligands to the thyroid hormone receptor. This hormone:receptor complex interacts with a specific DNA motif, called site A within the APO A1 gene to activate transcription of the gene to produce more APO A1 mRNA, which in turn leads to higher levels of the protein (Romney et al, 1992 and Taylor et al., 1996). Subsequent studies of thyroid hormone regulated actions of site A (−208 to −193) by thyroid hormones has led to the term thyroid hormone response element. In the absence of Site A, a negative effect can be observed which is mediated by a negative thyroid hormone response element at position −25 to −20 (Taylor et al., 1996). Our laboratory demonstrated that site-directed mutagenesis of the negative thyroid response element abolished the inhibitory effects of the hormone and increased basal promotor activity by up to 40-fold. Similar deletion studies have localized the inhibitory effects of estrogen receptor and 17 β-estradiol on rat APO A1 gene activity to the promotor element at Site B (−170 to −144) with Site S (−186 to −171) acting as an amplifier (Taylor et al., 2000).

Ischemic heart disease has a high incidence in the world and a substantial adverse impact on society. It is another aspect of the present invention to provide new ways to lower the risk of the disease. The fact that each year tens of billions of dollars are devoted to the delivery of healthcare for patients with ischemic heart disease alone demonstrates a continuing need to effectively prevent the disease.

U.S. Pat. No. 6,022,901 discloses the use of resveratrol to prevent or treat restenosis following coronary intervention. The method involves administration of an active agent comprising cis-resveratrol, trans-resveratrol, a mixture thereof, or a pharmacologically acceptable salt, ester, amide, prodrug or analog thereof. Related U.S. Pat. No. 6,211,247 claims a pharmaceutical composition for preventing or treating restenosis in an individual following coronary intervention.

U.S. Pat. No. 6,048,903 discloses a treatment for blood cholesterol with trans-resveratrol which has the effect of increasing the blood level of HDL and decreasing the blood level of LDL for reducing the risk of hypercholesterolemia.

U.S. Pat. No. 6,203,818 discloses a nutritional supplement for cardiovascular health via aiding in preventing, delaying the onset of and/or slowing the progression of atherosclerosis and coronary heart disease. The nutritional supplement comprises quercetin and folic acid or folate and additionally contains a flavanoid.

It is yet another aspect of the present invention to provided an increased understanding of the mechanisms of action to resveratrol and to provide a basis for the development of analogues that have similar beneficial actions.

It is still another aspect of the present invention to provide a molecular target for further drug development aimed at increasing APO A1 and/or HDL levels.

### REFERENCES

Barter P J & Rye K A. Molecular mechanisms of reverse cholesterol transport. Current Opinion in Lipidology 7:82-87, 1996
Chanvitayapongs S, Draczynska-Lusiak B, Sun A Y. Amelioration of oxidative stress by antioxidants and resveratrol in PC12 cells. Neuroreport 8:1499-1502, 1997.
Franceschini G, Maderna P & Sirtori C R. Reverse cholesterol transport: physiology and pharmacology. Atherosclerosis 88:99-107, 1991
Frankel E N, Waterhouse A L, Kinsella J E. Inhibition of human LDL oxidation by resveratrol. Lancet 341:1103-1104, 1993.
Jang M and others. Cancer chemopreventive activity of resveratrol, a natural product derived from grapes. Science 275:218-220, 1997
Luoma P V. Gene activation, apolipoprotein A-I/high density lipoprotein, atherosclerosis prevention and longevity. Pharmacology & toxicology 81 57-64, 1997
Miller N E. Associations of high-denisty lipoprotein subclasses and apolipoproteins with ischemic heart disease and coronary atherosclerosis. American Heart Journal 113 589-597, 1987.
Miller N E., Laville A & Crook D. Direct evidence that reverse cholesterol transport is mediated by high-density lipoprotein in rabbit. Nature 314 109-111, 1985.
Murao K., Wada Y., Nakamura T., Taylor A H., Mooradian A D & Wong N C. Effects of glucose and insulin on rat apolipoprotein A-1 gene expression. J Biol. Chem. July 24; 273 (30): 188959-65, 1998.
Rotondo S et al. Effect of trans-resveratrol, a natural polyphenolic compound, on human polymorphonuclear leukocyte function. British Journal of Pharmacology 123:1691-1699, 1998.
Soleas G J, Diamandis E P, Goldberg D M. Resveratrol: A molecule whose time has come? And gone? Clinical Biochemistry 30:91-113, 1997.
Taylor A H, Wishart P, Lawless D E, Raymond J, Wong N C. Identification of functional positive and negative thyroid hormone-responsive elements in the rat apolipoprotein A1 promoter. Biochemistry 1996 Jun. 25;35(25):8281-8.
Taylor A H., Fox-Robichaud A E, Egan C., Dionne J., Lawless D E., Raymond J., Romney J., Wong N C. Oestradiol decreases rat apolipoprotein A1 transcription via promotor site B. J. Mol. Endocrinol. 2000 Oct. 25(2): 207-19
Zheng X L., Matsubara S., Diao C., Hollenberg M D, & Wong N C. Activation of apolipoprotein A1 gene expression by protein kinase A and kinase C through transcription factor, Sp1. J Biol. Chem. October 13; 275(41): 31747-54 (2000)

All references cited herein are fully incorporated by reference.

### SUMMARY OF INVENTION

In accordance with the various aspects and principles of the present invention there are provided new tools and reagents for assaying and identifying compounds which can increase HDL levels by promoting APO A1 gene expression. Various regions related to the APO A1 gene and specifically within the relevant promoter region have been identified that appear to be important for controlling gene activity. Polyphenol compounds such as resveratrol have been discovered to enhance activity of the gene. Cell lines have been discovered and created which are useful as screening tools for identifying other such compounds including mimetics and analogs of resveratrol for upregulating APO A1 gene expression. Similarly, such tools can be advantageously employed to screen synthetic compounds or neutraceuticals for identifying those compounds capable of providing similar benefit on APO A1 expression.

A preferred embodiment involves methods for increasing HDL/APO A1 levels in plasma in an individual by administering therapeutically effective amount of an activating agent for selectively promoting APO A1 expression in intestinal and liver cells. Such activating agent acts upon the DNA within the intestinal cells, specifically at a DNA motif spanning −190 to −170 of the gene. It has been discovered that resveratrol or analogs thereof can act as such activating agents. Most preferred embodiments of such compounds will also comprise a pharmaceutically acceptable carrier such as a buffer, or other vehicle well known in the art.

Another preferred embodiment involves promoting APO A1 expression especially in intestinal cells.

Still other embodiments involve methods for identifying other genes that may be sensitive to resveratrol comprising incubating such genes with a complementary sequence of the motif within the APO A1 prromotor that is acted upon by resveratrol under hybridizing conditions and then assaying for the presence of hybridization of the complementary sequence of the motif promotor.

Yet another preferred embodiment involves screening for, and identifying, synthetic compounds or neutraceuticals that may increase circulating APO A1/HDL levels in mammals. The preferred procedure for screening or identifying candidate compound(s) involves exposing permanently transfected cells Hep G2 or CaCO2 cell lines to the synthetic compounds or neutraceuticals to be screened and assaying for elevated levels of APO A1 gene transcription and/or APO A1 protein whereby such elevated transcription levels or APO. A1 protein levels identify compounds or neutraceuticals capable of increasing circulating HDL levels. Other compounds for increasing APO A1 expression could similarly be identified by incubating such compounds with permanently transfected cell lines containing full or truncated APO A1 promotor sequences and assaying for increased APO A1 expression. The thusly identified compounds, particularly with pharmaceutically acceptable carriers would provide great clinical advantage.

### BRIEF DESCRIPTION OF THE FIGURES

Greater understanding of the principles of the present invention will be had by study of the accompanying figures wherein:

FIG. 1 shows a schematic map of the constructs in the transfection assays;

FIG. 2 shows the effects of resveratrol (0, 2.5, 5, 7.5 and 10 μM) on APO A1 promoter activity levels in CaCo2 cells transfected with pAI.474-Luc;

FIG. 3 shows the time course over which resveratrol (5 μM) had an effect on APO A1 levels in CaCO2 cells transfected with a reported construct, pAI.474-Luc. This construct pAI.474-Luc contained the rat APO A1 promoter DNA spanning −474 to −7 fused to the reporter gene, firefly luciferase (Luc). A significant effect was observed at 4, 8, 16 and 24 hours following administration of resveratrol but maximal stimulation appeared following 16 hours of exposure to the compound;

FIG. 4 shows a study in CaCO2 cells transfected with different reporter constructs that contained progressively smaller fragments of the APO A1 promoter and treated with 5 μM resveratrol for 16 hours. The number at the bottom of each set of columns denotes the 5′ location of the fragment and the 3′ end is common to all deletional clones at −7. For example, the left set of columns shows activity of the −474 to −7 fragment in the presence and absence of resveratrol, respectively. These results demonstrate that removal of the DNA from −190 to −171 of the promoter abolishes the response to resveratrol;

FIG. 5 shows a western blot analysis of APO A1 protein. This technique was used to measure the APO A1 protein content in spent media from cells untreated or treated with 5 or 10 μM of resveratrol for 36 hours;

FIG. 6 shows the results of Hep G2 cells transiently transfected with pAI.474-Luc and then treated with various doses of resveratrol for 16 hours. Cells treated with 0, 5, 10, 25, 50, 75 and 100 uM resveratrol showed a dose-response relationship with peak dose at 5 to 10 uM, but becoming inhibitory at 50 uM and above. These data have been normalized to β-gal (co-transfected reporter to control for transfection efficiency) and expressed relative to the protein levels. The experiment was repeated 3 times with 3 different batches of cells;

FIG. 7 shows data from HepG2 cells permanently transfected with pAI.474-Luc and a commercially available neomycin resistance gene. The cells from this transfection were selected for neomycin resistance. The cells that were neomycin resistant and had Luc-activity were retained for the studies because they contain both the pAI.474-Luc and the neomycin resistance marker. These cells were treated with resveratrol (0 to 25 μM). To create the permanently transfected cells, 474-Luc was co-transfected with another plasmid carrying neomycin resistance. The ability to grow in neomycin was a marker for successful transfection. A dose-response effect to resveratrol was observed with results mimicking that of transiently transfected cells;

FIG. 8 shows the time course of the APO A1 promoter response to resveratrol in Hep G2 cells transfected with the pAI.474-Luc, exposed to 10 μM of resveratrol, and then harvested at 4, 8, 16 and 24 hrs after exposure. The Luc-activity was assayed in the cells and results showed that maximal stimulation of the promoter began at 16 and extended to 24 hrs; and

FIG. 9 shows a western blot analysis to measure the APO A1 protein content in spent media from Hep G2 cells untreated or treated with 5 or 10 μM of resveratrol.

### DETAILED DESCRIPTION OF THE INVENTION

In accordance with principles of the present invention, a preferred embodiment describes a method for promoting APO A1 expression and characterizes the steps and potential mechanism in detail regarding the use of resveratrol to enhance transcription of the gene. Understanding its potential action will lead to improved development or searches for derivatives and analogues with enhanced therapeutic effect.

It is clear from the epidemiologic studies that cardiovascular disease (CVD) correlates with many parameters, but one of the most important is low levels of HDL/APO A1. Methodology that increases APO A1/HDL should reduce the risk of CVD. While hormonal regulation of APO A1 gene activity could be a way to control expression of the gene, an unfortunate accompanying disadvantage is that it is not possible to use increased concentrations of the hormones, such as thyroid hormone to up-regulate activity of the gene. Levels of thyroid hormone that exceed normal values are toxic in humans and therefore cannot be used to enhance APO A1 gene activity. Accordingly, the use of mimetics or analogues that can enhance APO A1 gene activity without the accompanying toxic effects is desired.

Experiment 1. Resveratrol treatment of CaCO2 cells, from intestine. This study determined whether resveratrol had an effect on APO AI gene in CaCO2 cells, an intestinal cell line. Cells were grown under conditions recommended by the ATCC and summarized briefly in the methods section. The initial studies examined the potential effects of resveratrol to increase APO A1 expression using histologic analysis. Cells were treated with 5 or 10 μM of resveratrol and then stained for their abundance of APO AI using a commercially available human APO A1 antibody (data not shown). The CaCO2 cells were examined using phase contrast and immunohistochemical staining of APO A1 protein in the absence (untreated) and presence of resveratrol (5 and 10 μM). Resveratrol caused an increase in the abundance of APO A1 signal following exposure to 5 and 10 μM of the agent after 36 hours of treatment. An increase in the level of APO A1 protein expression in the presence of resveratrol was also demonstrated. The results showed that both 5 and 10 μM of resveratrol increased the fluorescence arising from cellular content of APO A1 protein.

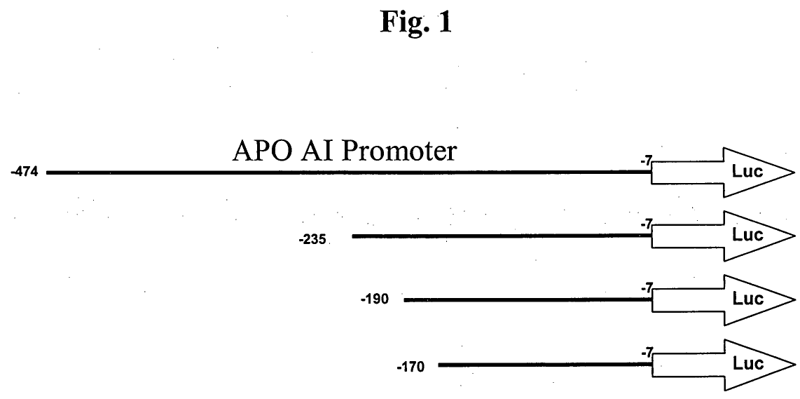

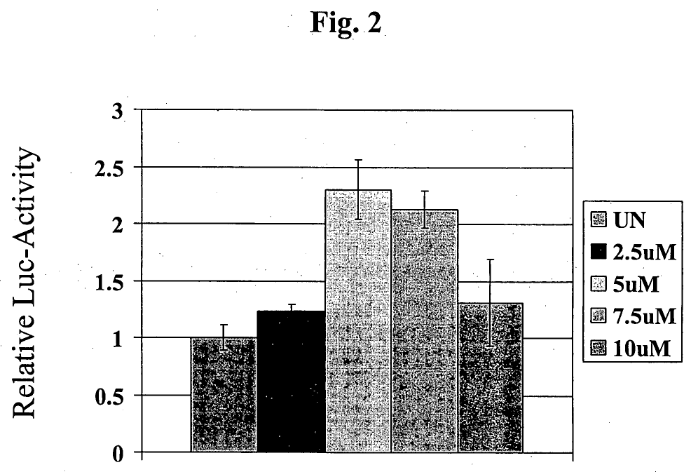

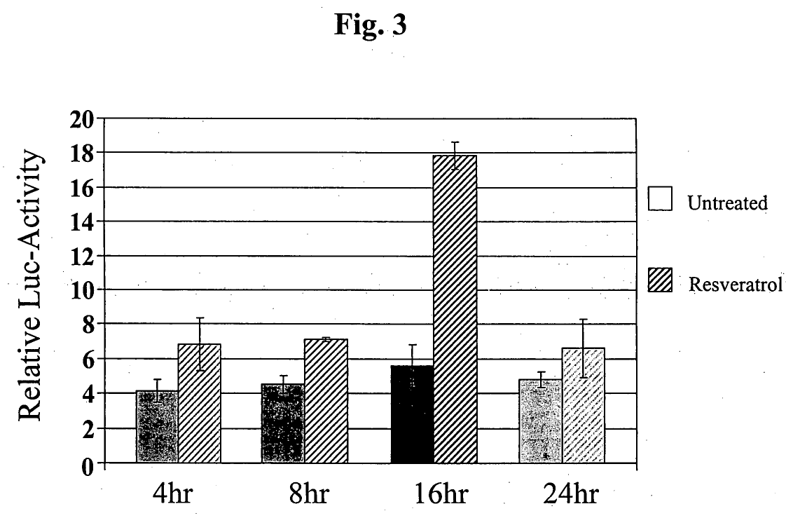

Next the CaCO2 cells were exposed to varying concentrations of resveratrol from 0 to 15 μM. The cells were transfected, using a standard technique, with the reporter construct, pAI.474-Luc (see map, FIG. 1) along with pRSV-β-galactosidase as a monitor for transfection efficiency. The pAI.474-Luc is a construct that we have created using conventional molecular biology techniques and contains the rat APO AI promoter from −474 to −7 fused to the reporter, firefly luciferase (Luc). The resveratol was dissolved in DMSO and then added to the culture media to yield a final concentration that varied from 0 to 15 μM. The cells were treated with the varying concentrations of the resveratrol for 16 hours. At the end of the treatment, the cells were harvested and the Luc-activity measured. These values were normalized to both lysate protein concentration and also β-galactosidase activity. The results (FIG. 2) showed that the resveratrol stimulated APO AI promoter activity maximally by 2.5-fold at a resveratrol concentration that ranged from 5 to 7.5 μM.

Whereas, the preceding studies showed that the resveratrol concentration, which caused maximal stimulation of the APO AI promoter activity ranged between 5-7.5 μM, the duration of action was unclear. In order to address this point, the same experiment to that above was used to assess the kinetics of resveratrol induction of the APO AI promoter. CaCO2 cells transfected with pAI.474-Luc were treated with 5 μM of resveratrol at selected time points varying from 4 to 24 hours. Results (FIG. 3) showed that the optimal time point for the stimulatory effects of resveratrol on the APO AI promoter appeared to be around 16 hours. The information arising from these studies show that resveratrol can stimulate APO AI gene transcription in CaCO2 cells and the time of maximal effect for resveratrol is roughly 16 hours after exposure.

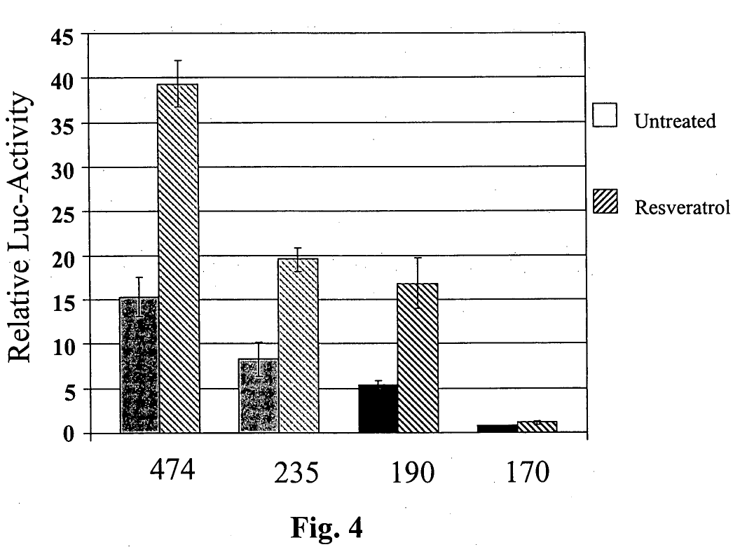

Experiment 2. Effects of resveratrol require a fragment of the DNA spanning nucleotides −190 to −170. Since pAI.474-Luc, used in the above studies, was found to mediate effects of resveratrol and this construct contained the rat APO AI DNA fragment spanning −474 to −7, we postulated that a motif or motifs within this segment of the promoter DNA mediates actions of the compound. In order to identify the potential motif(s), separate constructs containing progressively smaller amounts of APO AI DNA were fused to the Luc gene. The activity of each construct was tested by transient transfection assay in CaCO2 cells and then treated with 5 μM resveratrol for a minimum of 16 hours. Results (FIG. 4) showed that the full-length (474 to −7) promoter produced a 2.5-fold induction. Removal of the DNA the −235 or −190 to −7 fragments from the parent promoter did not affect the ability of resveratrol to induce the 2.5-fold increase in promoter activity. In contrast, further deletion with the remaining −170 to −7 fragment of the promoted abolished the resveratrol induction of the promoter. We discovered the resveratrol responsive motif in the APO AI DNA must span nucleotides −190 to −170.

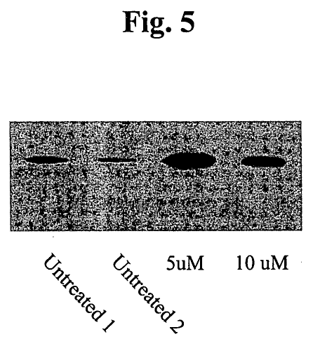

Experiment 3. Resveratrol increases APO AI protein secreted from CaCO2 cells. This experiment sought to measure whether resveratrol stimulation of transcriptional activity of the promoter in the CaCO2 cells increased the abundance of the APO AI protein, ultimately responsible for the antiatherogenic activity of the gene. Resveratrol increased activity of the APO AI promoter in the pAI.474-Luc construct, a transgene that is introduced into CaCO2 cells by transient transfection but whether it affected activity of the APO AI gene endogenous to the CaCO2 cells was not known. For these studies, CaCO2 cells were cultured as usual and exposed to media containing resveratrol at a concentration of 5 or 10 μM for 36 hours. Llonger exposure of the cells to resveratrol was utilized to allow adequate time for the APO AI protein to be secreted into the media from the CaCO2 cells, and detected. Spent media exposed to the cells for 36 hours was assayed for its content of APO AI protein using western blott analysis. Results (FIG. 5) showed a marked increase in abundance of APO AI protein in the spent media from cells treated with resveratrol but APO AI in the media lacking resveratrol was lower.

The results of these studies show that the antiatherogenic properties of resveratrol augments expression of the APO AI gene. Increased expression of the APO AI gene augments RCT and thereby enhances the removal of cholesterol from the body. The data in CaCO2 cells are significant and we have unexpectedly:
1.) Identified for the first time effects of resveratrol on APO AI in intestinal cells.
2.) Identified that resveratrol affects transcription of the APO AI gene.
3.) Determined the time required for resveratrol to act on APO AI in the cells.
4.) Determined the range of resveratrol concentration to therapeutically alter APO AI gene expression.
5.) Identified the DNA motif that mediates resveratrol effects in CaCO2 cells.
6.) Showed that one effect of resveratrol is to increase abundance of APO AI protein.

This information will be useful in harnessing the of use of resveratrol or other similar APO A1 increasing agents by:
1.) Designing a formulation of resveratrol that may be released into the intestine.
2.) Designing a formatulation for timed release of resveratrol or such agents to insure that it will be in the intestinal track for a minimum of 16 hours.
3.) Designing a formulation for maintaining presence of a therapeutic dose of resveratrol or such agents that was not previously known.
4.) Demonstrating use of various reporter constructs and cell lines for assaying the actions of resveratrol or such agents and extending it for screening of natural or synthetic polyphenols or other agents similar in action to that of resveratrol.

Experiment 4. Resveratrol treatment of Hep G2 cells, from liver. Since the APO AI gene is expressed in both liver and small intestine, the following studies examine the ability of resveratrol to affect expression of the gene in liver cells. The first set of studies examined the potential ability of resveratrol to increase the abundance of APO A1 and to assess this possibility using histological analysis. Cells were grown under conditions recommended by the ATCC and summarized briefly in the methods section. The initial studies examined the potential effects of resveratrol to increase APO A1 expression using histologic analysis. Cells were treated with 5 or 10 μM of resveratrol and then stained for their abundance of APO AI using a commercially available human APO A1 antibody. Hep G2 cells were viewed under phase contrast or fluorescence microscopy following treatment with or without resveratrol and immunostaining for their content of APO A1 protein. The results showed an increase in fluorescence for APO A1 signal following treatment with 5 or 10 uM of resveratrol.

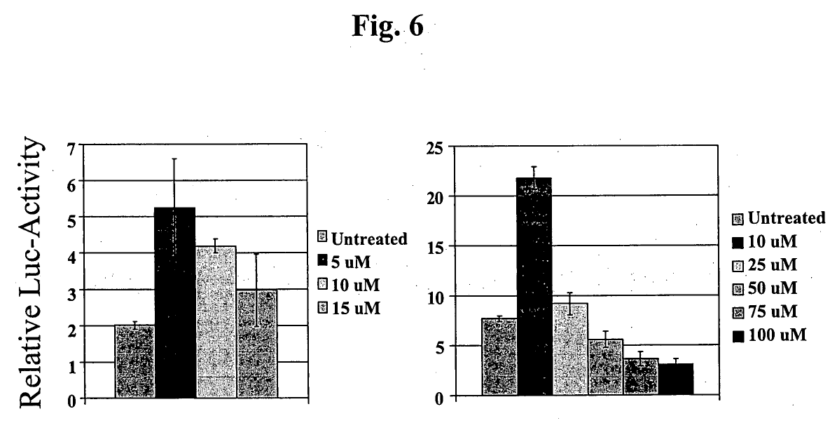

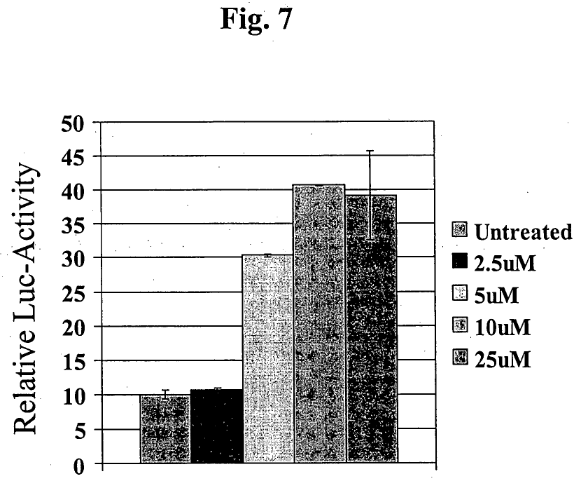

To assay for promoter activity in Hep G2 cells, the reporter construct pAI474-Luc was inserted into the human hepatoma, Hep G2, cells along with pRSV-β-galactosidase as a monitor for transfection efficiency using conventional molecular biology techniques as later described. The transfected cells were exposed to varying concentrations of resveratrol from 0 to 100 μM for 16 hours. The cells were harvested and assayed for Luc-activity. The values obtained were normalized relative to both protein and β-galactosidase activity. Results (FIG. 6) showed a 3-fold increase in activity following treatment with 5 to 10 μM resveratrol. However, further increases in the concentration of resveratrol did not further increase Luc-activity of the reporter construct and in fact, concentrations of the compound at 15, 25, 50, 75 or 100 μM were associated with no significant increases but rather led to a decrease of 50% in Luc-activity. To verify these observations, a cell line was created that contained the pAI.474-Luc permanently inserted into the cells. These permanently transfected cells were tested for response to resveratrol concentrations ranging from 0-20 μm. Results (FIG. 7) showed that Luc-activity in the permanently transfected cells increased in a dose dependent fashion with a maximal increase of 4-fold following treatment with 10 μM resveratrol.

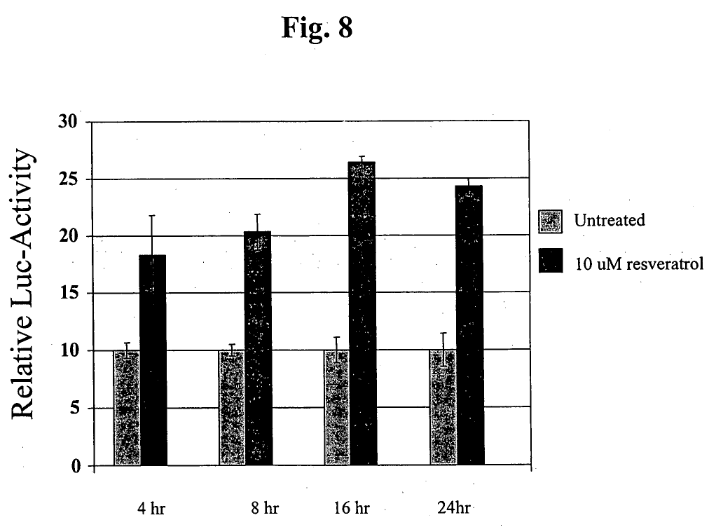

The time course of pAI.474-Luc was tested in response to a fixed concentration of resveratrol. In this study Hep G2 cells were transiently transfected with pAI.474-Luc and then exposed to 10 μM resveratrol. The cells were harvested at 4, 8, 16 and 24 hours. The maximal effect of the resveratrol was similar to that in the CaCO2 cells with maximal increase observed after 16 hours of treatment (FIG. 8).

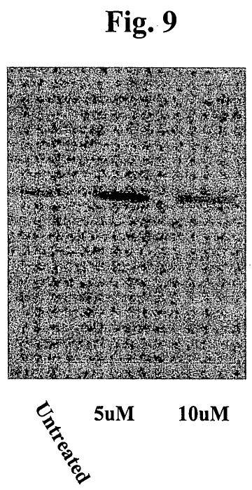

Experiment 5. Resveratrol increases APO AI protein secreted from Hep G2 cells. To measure whether resveratrol stimulation of the APO AI promoter in the Hep G2 cells also increases the abundance of the protein, APO AI secreted into the media was assessed following treatment with the compound. Resveratrol increased the activity of the APO AI promoter in the pAI.474-Luc construct, a transgene that was introduced into Hep G2 cells by transient or stable transfection. Hep G2 cells were cultured as usual and exposed to media containing resveratrol at a concentration of 5 or 10 μM for 36 hours. Spent media exposed to the cells for 36 hours were assayed for its content of APO AI protein using western blot analysis. Results (FIG. 9) showed a marked increase in abundance of APO AI protein in the spent media from cells treated with resveratrol but APO AI in the media lacking resveratrol was lower.

These experiments demonstrate that resveratrol also unexpectedly and advantageously increased expression of the APO AI gene in Hep G2 cells derived from liver. A preferred embodiment of a screening assay would therefore advantageously contain a permanently transfected Hep G2 cell line containing the pAI.474-marker where a preferred marker is Luc. Such cells could be used to screen for compounds or agents for increasing APO A1 expression or transfection. The experiments show the preferred time periods for therapeutic application of such compounds as well as how the preferred therapeutic concentrations may be initially determined. Of course, it will be readily recognized that conventional clinical trials are needed to refine therapeutic regimens in accordance with their purpose.

We have discovered resveratrol to advantageously affect the expression of the Apo A1 gene. Using human cell lines, Hep G2 and CaCO2, an increase in levels of Apo A1 protein and promotor activity in both cell types exposed to resveratrol concentrations in the range of 5-10 uM was observed. Equally important is that exposure of cells to concentrations that exceed this range has a detrimental effect on expression of the Apo A1 gene. In addition, the finding that gene activity in response to a single exposure of resveratrol had maximal effect on transcription of the gene at 16-24 hours but levels of the protein could be detected up to 36 hours after exposure is also new information that guides determination of the length of time required for exposure of the cells to resveratrol for therapeutic effect. The fact that CaCo2 derived intestinal cells respond to resveratrol is also new. This fact is important because resveratrol will contact the intestinal cells first before going to the liver and therefore, the interaction and effect of resveratrol on intestinal cells is likely more important then its effect on liver cells because the concentrations of resveratrol after consumption may never reach levels in the blood to sufficiently stimulate the liver cells.

In addition to these basic observations, the mechanism by which resveratrol stimulated Apo A1 gene transcription was tested in assays that employed deletional constructs of the promoter. These studies show that resveratrol in the CaCO2 cells act via the −190 to −170 fragment of DNA but the effect in liver cells may be due to interaction at the same or different site. This is important because in order to produce a beneficial effect in the intestinal cells using derivatives or analogues of resveratrol, it may be be different from that on the liver.

In another embodiment of this invention, permanently transfected HepG2 cells are used as a screening system to screen for the resveratrol sensitive promotor sequence in other genes. Permanently transfected HepG2 or CaCO2 cells with deletional constructs can provide the basis of an assay system for screening of resveratrol sensitive promotor sequences in genes, and for screening neutraceuticals and pharmaceuticals to identify those that may regulate Apo A1 expression. With additional reference to the figures and the legend descriptions provided above: the following procedures are provided

Methodology

Cell Culture

Human hepatoblastoma cells (HepG2) and intestinal cells (CaCo2) were obtained from the American Type Culture Collection (Rockville, Md.). Cells were grown in Minimum Essential Medium (MEM) (Gibco) supplemented with 2 mM glutamine, MEM vitamin solution and 10% fetal bovine serum (FBS) for HepG2 and 20% FBS (Gibco) for CaCo2 cells. All cells were incubated in a 95% air/5% CO₂ atmosphere.

Plasmids

The plasmids created for the studies contained the rat APO A1 promoter from −474, −375, −325, −235, −190 to −170 fused to the firefly luciferase gene in the vector, pGL3 (Promega). Insertion of the promoter DNA was verified by nucleotide sequence analysis. Plasmid DNA was prepared from bacteria containing the desired clone and isolated using Qiagen kits according to manufacturer's instructions and used in the transfection studies or to create a stable cell line.

Cell Treatments

The CaCo2 or HepG2 cells were grown in the defined media and, for promoter assay studies, transfected with the reporter construct of interest. Cells were then left in serum-free media for 8-12 hours after which time resveratrol was added to media to give a final concentration of the agent as stated in the figure legends. The cells were exposed to the agent for varying periods of time, harvested and then the parameter of interest, either APO A1 protein or promoter activity, was assayed.

Transient/Permanent Transfections

For transient transfections cells were seeded onto six well plates and grown to 30-40% confluence. The cells were then transfected using 5 ul of Superfect (Qiagen) and up to one microgram of the plasmid of interest in 100 ul of serum and antibiotic free MEM. The solution was incubated for 10 minutes at room temperature. Media was then removed from the cells to be transfected and 1 ml of media was added to the DNA-Superfect mixture before being applied to the cells. The cells were then exposed to the DNA for 2 hours at 37° C./5% CO2 and then the media containing DNA was removed and replaced with serum free MEM media allowed to grow over night prior to harvest.

HepG2 cells were also permanently transfected with 474-luciferase using a co-transfection method. Hep G2 cells are grown in MEM (Gibco) and 10% fetal calf serum (Gibco) and then co-transfected with 474-Luc along with another plasmid that carries neomycin resistance. Then 400-600 ug per ml of neomycin was added to the media and the cells surviving treatment with neomycin assayed for Luc-activity, which when present demonstrates the cells have been permanently transfected.

Preparation of Cell Lysate for Luciferase and Beta-Galactosidase Assays.

Cells were transfected with CAT plasmid of interest (see above) along with 0.5 ug of Rous sarcomavirus-B-galactosidase, RSV-beta-Gal to monitor the efficiency of DNA uptake by cells. All cells were then left in serum poor media for 12 hours before treatment with resveratrol (Calbiochem) for various periods of time. Harvested cells were then lysed using a commercially available reporter lysis buffer (Promega) and cellular debris was collected at 13,000 rpm for 5 minutes. Aliquots of the supernatant were taken for measurement of B-galactosidase activity (Promega) and for total protein determination using Bradford Assay (Bio-Rad reagent).

Measurement of Luciferase Activity

Cells were transfected with Luciferase plasmid of interest (see above) and left to recover overnight in serum poor media. These cells or those that were permanently transfected with the luciferase promoter were then treated with varying concentrations of resveratrol for stated periods of time. As above, RSV-beta-Gal was co-transfected as a control to normalize for DNA uptake. Cells were then harvested and suspended in reporter lysis buffer (Promega). A 10 ul aliquot of this lysate was used for determination of luciferase activity, and 5 ul were used for total protein determination (Bradford Assay, Bio-Rad reagent). Luciferase activity was then determined and expressed relative to the protein concentration of that sample.

Western Blotting

Media or cells were harvested from untreated and treated HepG2/CaCo2 culture dishes at various time points and stored at −80C when required. For experiments in which media was collected for western blotting, cells from these dishes were trypsinized (Gibco) and a 100 ul sample of cells was used to determine the percentage of dead cells by counting live/dead cell ratios using coomasie blue staining. The remaining cells were then assessed for total DNA content using method described by Maniatis, (Cloning Manual). DNA content per dish was then utilized along with ratio of live/dead cells to normalize the amount of media to be separated by polyacrylamide gel electrophoresis. For experiments requiring western blot of whole cell lysates, cells were harvested and lysed using reporter lysis reagent (Promega) and cell debris was spun down at 13,000 rpm for 5 minutes. An aliquot of the supernatant was then used to determine amount of protein per sample using Bradford assay (Bio-Rad reagent). Equal amounts of protein from all samples were then separated by polyacrylamide gel electrophoresis as was done with media. The gels were then transferred to nitrocellulose membrane (Hybond, Amersham Pharmacia Biotech), which was then probed with a monoclonal antibody against human ApoA1 (Calbiochem).

Immunofluorescence Labeling of Apo A1

HepG2 and CaCo2 cells were grown on cover slips. Cover slips on which CaCo2 cells were grown were also coated with fibronectin (Calbiochem). After treatments with various amounts of ethanol or resveratrol for 24 or 48 hours, the cells were fixed and permeabilized with a solution containing a mixture of 37% formaldehyde, 0.25% glutaraldehyde and 0.25% triton-X in PEM buffer (160 mmol/L PIPES, 10 mmol/L egtazic acid (EGTA), 4 mmol/L MgCl2, pH 6.9) for ten minutes at room temperature. After washing three times with phosphate-buffered saline (PBS) the cells were treated with the reducing agent sodium borohydride, 1 mg/ml in PBS for 3×5 minutes. The cells where then washed again in PBS. Mouse monoclonal anti-APO A1 antibody (Calbiochem) was diluted 1:50 with PBS and added to each coverslip and incubated in a humid chamber for 60 minutes at room temperature. After washing, the FITC-conjugated secondary antibody (goat anti-mouse IgG, Jackson ImmunoResearch) was diluted 1:200 with PBS and added to coverslips for 45-60 minutes at room temperature. Cells were then given a final wash with PBS and mounted on glass slides using mounting media containing P-phenylene diamine and 50% glycerol in PBS. The FITC-labeled ApoA1 peptide in cells was visualized using a Zeiss fluorescence microscope (Zeiss, Dusseldorf, Germany) with FITC excitation and emission wavelengths of 488 and 520 nm. Photographs were taken using a Kodak digital camera mounted onto the microscope. Exposure times were identical for both treated and untreated cells. Final magnification was 250×.

1-13. (canceled)

14. A method for screening and identifying a compound that is capable of increasing plasma HDL levels in a mammal, comprising:
a) exposing cells to the compound to be screened; and
b) determining whether the compound acts upon the −190 to −170 DNA motif of the APO AI gene, wherein the +1 position is the transcription start of the gene, thereby increasing APO AI gene expression,
thereby identifying a compound capable of increasing plasma HDL levels.

15. The method of claim 14, wherein the compound is a resveratrol analog and/or mimetic.

16. The method of claim 14, wherein the cells are Hep G2 cells.

17. The method of claim 14, wherein the cells are CaCO2 cells.

18. The method of claim 14, wherein the cells are permanently transfected with a plasmid comprising a promoter sequence comprising the −190 to −170 DNA motif of the APO AI gene operably linked to a reporter gene.

19. The method of claim 18, wherein the cells are Hep G2 cells.

20. The method of claim 18, wherein the cells are CaCO2 cells.

21. The method of claim 18, wherein the promoter sequence is a full or truncated APO AI gene promoter sequence.

22. The method of claim 18, wherein the plasmid is pAI.474.

23. The method of claim 18, wherein the reporter gene is Luciferase.

24. The method of claim 18, wherein APO AI gene expression activity is measured at least about 16 hours after exposing the cells to the compound.

25. The method of claim 14, comprising measuring APO AI gene expression activity by assaying for levels of Apo AI protein.

26. The method of claim 25, wherein assaying for levels of Apo AI protein comprises using Western blotting.

27. The method of claim 25, wherein the levels of Apo AI protein are measured at least about 36 hours after exposing the cells to the compound.

28. The method of claim 18, further comprising exposing a separate population of the cells to the compound.

29. The method of claim 28, further comprising measuring APO A1 gene expression activity by assaying the separate population of the cells for levels of Apo AI protein.

30. The method of claim 29, wherein assaying for levels of Apo Al protein comprises using Western blotting.
